(19) United States
(12) Patent Application Publication
(10) Pub. No.: US 2003/0044468 A1
(43) Pub. Date: Mar. 6, 2003

(54) Two-phase processing of thermosensitive polymers for use as biomaterials

(76) Inventors: Francesco Cellesi, Zurich (CH); Nicola Tirelli, Uster (CH); Jeffrey A. Hubbell, Zurich (CH)

Correspondence Address: CLARK & ELBING LLP, 101 FEDERAL STREET, BOSTON, MA, 02110, US

(21) Appl. No.: 10/102,247

(22) Filed: Mar. 20, 2002

Related U.S. Application Data
(60) Provisional application No. 60/277,513, filed on Mar. 20, 2001.

(51) Int. Cl. A61K 9/00; C12N 5/08; C08F 220/06

(52) U.S. Cl. 424/487; 435/357; 435/358; 525/329.5

(57) ABSTRACT

A two-step system for preparing biomaterials from polymeric precursors is disclosed. The method involves (a) shaping the polymeric precursors by inducing thermal gelation of an aqueous solution of the polymeric precursors and (b) curing the polymeric precursors by cross-linking reactive groups on the polymeric precursors to produce a cured material. The curing reaction involves either a Michael-type addition reaction or a free radical photopolymerization reaction in order to cross-link the polymeric materials. The biomaterials produced by this method have a variety of biomedical uses, including drug delivery, microencapsulation, and implantation.

### CROSS-REFERENCE TO RELATED APPLICATION

This application claims priority to copending U.S. Provisional Application No. 60/277,513, filed Mar. 20, 2001, hereby incorporated by reference.

### BACKGROUND OF INVENTION

The invention relates to the field of methods for making polymeric biomaterials.

Synthetic biomaterials, including polymeric hydrogels and water-soluble copolymers, are used in a variety of biomedical applications, including pharmaceutical and surgical applications. They can be used, for example, to deliver therapeutic molecules to a subject, as adhesives or sealants, for tissue engineering and wound healing scaffolds, and for encapsulation of cells and other biological materials.

The use of polymeric devices for the release of pharmaceutically active compounds has been investigated for long term, therapeutic treatment of various diseases. It is important for the polymer to be biodegradable and biocompatible. In addition, the techniques used to fabricate the polymeric device and load the drug should be non-toxic, result in dosage forms that are safe and effective for the patient, minimize irritation to surrounding tissue, and be a compatible medium for the drug being delivered.

While much progress has been made in the field of polymeric biomaterials, further developments are needed in order for such biomaterials to be used optimally in the body. Ideally, techniques for preparing polymeric materials for use as encapsulation materials or for the controlled delivery of drugs, including peptide and protein drugs, should be very mild and gentle, be able to proceed in an aqueous environment, allow for a subsequent or simultaneous cross-linking for chemical and mechanical stability, and provide materials that are stable for a specified time under physiological conditions. Currently, there are few methods for generating polymeric materials that meet these stringent requirements. Many of the most commonly used polymers for such applications have problems associated with their physicochemical properties and method of fabrication. Thus, there is a strong need for improved polymeric biomaterials and methods for their preparation.

### SUMMARY OF INVENTION

The present invention features a method for preparing a biomaterial from a polymeric precursor. The method includes the steps of (a) providing a polymeric precursor, including reactive groups, that undergoes reverse thermal gelation in aqueous solution; (b) shaping the precursor by thermally inducing gelation of an aqueous solution of the precursor; and (c) curing the polymeric precursor by cross-linking the reactive groups to produce a biomaterial. The polymeric precursors are, for example, polyethers or block copolymers, with at least one of the blocks being a polyether, poly(N-alkyl acrylamide), hydroxypropylcellulose, poly(vinylalcohol), poly(ethyl(hydroxyethyl)cellulose), polyoxazoline, or a derivative containing reactive groups in one or more side chains or as terminal groups.

In one embodiment, the curing step involves cross-linking the polymeric precursor using a Michael-type addition reaction. For this reaction, the Michael-donor is, for example, a thiol or a group containing a thiol, and the Michael-acceptor is, for example, an acrylate, an acrylamide, a quinone, a maleimide, a vinyl sulfone, or a vinyl pyridinium.

Alternatively, the curing step involves a free radical polymerization reaction that occurs in the presence of a sensitizer and an initiator. The sensitizer is, for example, a dye, such as ethyl eosin, eosin Y, fluorescein, 2,2-dimethoxy-2-phenyl acetophenone, 2-methoxy, 2-phenylacetophenone, camphorquinone, rose bengal, methylene blue, erythrosin, phloxime, thionine, riboflavin, methylene green, acridine orange, xanthine dye, or thioxanthine dyes. Exemplary initiators include triethanolamine, triethylamine, ethanolamine, N-methyl diethanolamine, N,N-dimethyl benzylamine, dibenzyl amine, N-benzyl ethanolamine, N-isopropyl benzylamine, tetramethyl ethylenediamine, potassium persulfate, tetramethyl ethylenediamine, lysine, ornithine, histidine, and arginine.

In a related aspect, the invention features physiologically compatible gels prepared by the above methods. The gels can be prepared in such forms as capsules, beads, tubes, hollow fibers, or solid fibers. The gels may also include a bioactive molecule, such as a protein, naturally occurring or synthetic molecules, viral particles, sugars, polysaccharides, organic or inorganic drugs, and nucleic acid molecules. Cells, such as pancreatic islet cells, human foreskin fibroblasts, Chinese hamster ovary cells, beta cell insulomas, lymphoblastic leukemia cells, mouse 3T3 fibroblasts, dopamine secreting ventral mesencephalon cells, neuroblastoid cells, adrenal medulla cells, and T-cells, may also be encapsulated in the gels of the invention.

In another aspect, the invention features drug delivery vehicles that include gels prepared by the above methods and therapeutic substances. The invention further provides a method for delivering a therapeutic substance to an animal, e.g., a human, that involves contacting a cell, tissue, organ, organ system, or body of the animal with this delivery vehicle. The therapeutic substance can be, for example, a prodrug, a synthesized organic molecule, a naturally occurring organic molecule, a nucleic acid, e.g., an antisense nucleic acid, a biosynthetic protein or peptide, a naturally occurring protein or peptide, or a modified protein or peptide.

Other features and advantages of the invention will be apparent from the following detailed description thereof and from the claims.

By “antisense nucleic acid” is meant a sequence of nucleic acid that is complementary to and binds to a sense sequence of nucleic acid, e.g., to prevent transcription or translation.

By “bioactive molecule” is meant any molecule capable of conferring a therapeutic effect by any means to a subject, e.g., a patient.

By “biomaterial” is meant a material that is intended for contact with the body, either upon the surface of the body or implanted within it.

By “conjugation” or “conjugated” is meant the alternation of carbon- carbon, carbon-heteroatom, or heteroatom-heteroatom multiple bonds with single bonds.

By “cured material” is meant a polymeric material that has undergone the shaping and the curing phases.

By “curing” or “curing phase” is meant the stabilization of a polymeric material through the cross-linking of reactive terminal or side groups. The curing phase of the invention is based on a chemical reaction, such as a Michael-type addition reaction or a free radical polymerization reaction.

By “initiator” is meant a molecule that, after electron transfer, generates a free radical and starts a radical polymerization reaction.

By “LCST” or “Lower Critical Solution Temperature” is meant the temperature at which a polymer undergoes reverse thermal gelation, i.e., the temperature below which the copolymer is soluble in water and above which the polymer undergoes phase separation to form a semi-solid gel. In desirable embodiments, the LCST for a polymer is between 10 and 90° C.

By “polymeric precursor” is meant a polymeric material that has not undergone a shaping or curing phase.

By “polymerization” or “cross-linking” is meant the linking of multiple precursor component molecules that results in a substantial increase in molecular weight. “Cross-linking” further indicates branching, typically to yield a polymer network.

By “prodrug” is meant a therapeutically inactive compound that converts to the active form of a drug by enzymatic or metabolic activity in vivo.

The terms “protein,” “polypeptide,” and “peptide” are used interchangeably herein and refer to any chain of two or more naturally occurring or modified amino acids joined by one or more peptide bonds, regardless of post-translational modification (e.g., glycosylation or phosphorylation).

By “reverse thermal gelation,” “thermal gelation,” or “thermally induced gelation” is meant the phenomenon whereby a polymer solution spontaneously increases in viscosity, and in many instances transforms into a semi-solid gel, as the temperature of the solution is increased above the LCST of the polymer.

By “sensitizer” is meant a chemical substance that through an interaction with UV and/or visible light generates a radical by electron exchange between its excited state and another molecule.

By “shaping” or “shaping phase” is meant a phase in the processing of a polymeric material in which the material is formed and shaped from a homogenous solution. The shaping phase of the present invention is based, for example, on a thermally induced gelation of an aqueous solution of the polymeric material.

### BRIEF DESCRIPTION OF THE DRAWINGS

FIG. 1 is a schematic diagram showing a free radical photopolymerization reaction.

FIG. 2 is a graph showing the change in the elastic and viscous modulus of a polymer solution with increasing temperature.

FIG. 3 is a pair of graphs showing the change in the elastic and viscous modulus of a polymer solution (subjected to curing without thermal gelation) over time.

FIG. 4 is a graph showing the change in the elastic and viscous modulus of a polymer solution (subjected to curing with thermal gelation) over time.

### DETAILED DESCRIPTION OF THE INVENTION

We have discovered that it is possible to form cured materials in the presence of sensitive biological materials by using highly selective curing reactions that are capable of proceeding under physiological conditions (such as Michael-type addition of thiols onto electron-poor olefins) and by using polymeric precursors that have negligible cytotoxicity. The mild character of the curing reactions allows for the incorporation of biological or bioactive molecules (e.g. peptides, proteins, nucleic acids, and drugs) into the polymeric materials, without adversely affecting the activity of these sensitive molecules. It also permits cells and cell aggregates to be successfully incorporated into the polymeric material.

Based on this discovery, we have developed a new processing technique for the preparation of biomaterials useful for cell encapsulation, controlled delivery of bioactive compounds, and implantation. The technique employs a two-step approach for producing biomaterials from polymeric precursors that involves (1) a shaping phase based on physical phenomena and (2) a curing phase that utilizes a chemical reaction to stabilize the polymeric material. In particular, the method involves the sequential use of reversible thermal gelation followed by chemical cross-linking by reaction of groups present in the polymeric material to produce a cured product. This method not only allows for the polymeric materials to be shaped with a conformal thermal treatment, but also makes it possible to tune the hydrophobicity and the hydrolytical degradation rate of the materials.

Processing and Structure of the Polymeric Precursors

The cured materials of the invention can be formed, for example, in commercial encapsulators. For encapsulation purposes, the shaping and curing phases are performed sequentially after the formation of regular droplets of the polymeric precursors, with or without biological material dispersed therein. The shaping and curing phases are performed in an appropriate bath where the drops are collected, preferably using a temperature difference between bath and dropping solution for the shaping phase and pH- or photo-activated reactions for the curing phase.

The shaping phase employs a phenomenon known as thermal gelation. A number of polymers have a solubility in water which is modified beyond a certain temperature point. These polymers exhibit a critical temperature, which defines their solubility in water. Polymers that have a Lower Critical Solubility Temperature (LCST) are soluble at low temperature (e.g., ambient temperature) but are not soluble above a higher temperature, i.e., below the LCST, the polymers are substantially soluble in the selected amount in the solvent, while above the LCST, solutions of this polymer form a multiphase system. This reverse solubility behavior leads to the phenomenon of thermal gelation, whereby an aqueous polymer solution spontaneously increases in viscosity, generally transforming into a semisolid gel, as the temperature of the solution is increased above the LCST of the polymer. By utilizing polymers that exhibit reverse thermal gelation, it is possible to shape the polymeric material by conformal thermal treatment.

The cured material of the invention is preferably made of polymers that are resistant to protein absorption, so as to limit inflammatory reactions when the material is implanted or otherwise comes in direct contact with living tissues. The polymeric precursors should have a Lower Critical Solubility Temperature (LCST) in water, i.e., a reversible gelation that occurs upon heating and is based on the release of water molecules structured around the chain of a polymer with limited hydrophilicity. Triblock copolymers of the Pluronic series (poly(ethylene glycol-bl-propylene glycol-bl-ethylene glycol)) or tetrablock copolymers of the Tetronic series provide convenient structure, because they are commercially available in a variety of compositions, are characterized by well-defined LCST, can be easily end-functionalized, and depending on the composition, show LCST in any desired temperature range between 10 and 90° C. Other polymer backbones, such as poly(N-isopropyl acrylamide) (PNIPAM) and other N-substituted acrylamides, poly(methyl vinyl ether), poly(ethylene oxide) (PEO) of convenient molecular weight, hydroxypropylcellulose, poly(vinylalcohol), poly(ethyl(hydroxyethyl)cellulose), and poly(2-ethyloxazoline), can be successfully used for this application, with the optional introduction of functional groups in the side chains via copolymerization (or as end groups in the case of PEO) (Scheme 1).

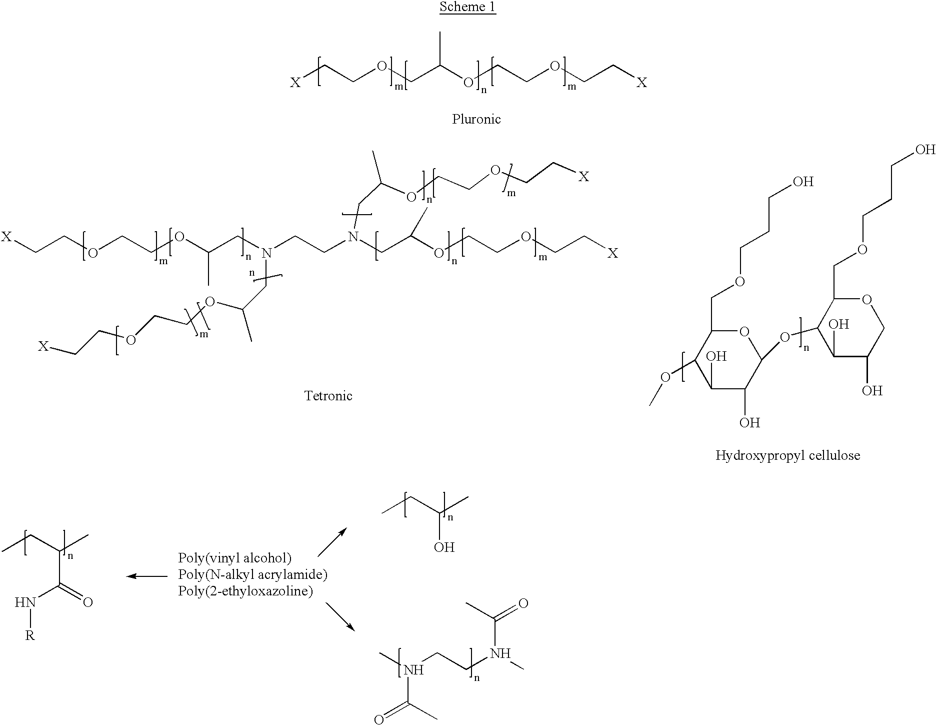

Exemplary LCST's are between 15 and 25° C. for solutions having a concentration of polymeric precursor of <20-25% w/w. This temperature range ensures that the polymeric precursors can be easily processed below the LCST without excessive freezing damage to the biological material dispersed therein. The polymer concentration of <20-25% w/w ensures that the cured material remains essentially water-based, keeps the viscosity of the aqueous solution of polymeric precursors low, and minimizes any potential cytotoxic effects.

Polymers with LCST behavior can be used as coating materials. In one embodiment of the invention, the polymeric precursors are used for conformal coating of, for example, the internal surface of tubing. In this embodiment, the shaping phase generates a layer of polymeric material through gelation of an aqueous solution of the polymeric precursors onto the tubing walls, which are maintained at a temperature above the LCST. A pH- or photo-activated reaction (curing phase) may follow to stabilize the coating.

Curing Reaction

After the shaping phase, the polymeric materials undergo a curing phase in order to provide mechanical and chemical stability. The curing phase increases stability by cross-linking reactive groups present in the polymeric materials. The curing reaction needs to proceed under physiological conditions, without the generation of toxic byproducts or causing other possible detrimental effects on cellular metabolism.

Accordingly, the curing phase of the invention uses either a Michael-type addition reaction, in which one component is a strong nucleophile and the other possesses a conjugated unsaturation, or a free radical photopolymerization reaction. Both of these types of reactions have been successfully used for the production of organic biomaterials in presence of cellular material (see, e.g., Hubbell et al., U.S. Ser. No. 09/496,231, filed Feb. 1, 2000; Hubbell et al., U.S. Pat. No. 5,858,746; and Hubbell et al., U.S. Pat. No. 5,801,033). These reactions produce a cross-linked material in the curing phase through the reaction of functional groups at the polymer ends or in the polymer side chains. As is explained below, the chemical structure of the reacting groups depends on the particular polymerization technique employed. With these reactions, a network can be generated with precise control over the distance between cross-links, and thus over the mechanical properties of the cured material, which depends primarily, if not exclusively, on the molecular weight of the polymeric precursors.

Michael-type Reactions

As previously discussed, one type of chemical reaction that can be used in the curing phase is a Michael-type reaction, which involves the 1,4 addition reaction of a nucleophile on a conjugated unsaturated system (Scheme 2).

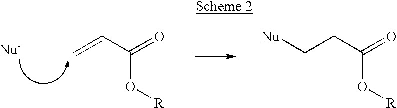

The nucleophilic components of this reaction are known as Michael-donors and the electrophilic components are referred to as Michael-acceptors. A suitable chemical reaction system utilizing a Michael-type reaction is described, for example, in U.S. Ser. No. 09/496,231, U.S. Ser. No. 09/586,937, filed Jun. 2, 2000, and U.S. Ser. No. 10/047,404, filed Oct. 19, 2001.

The advantage of this reaction system is that it allows for the production of cross-linked biomaterials in the presence of sensitive biological materials, such as drugs (including proteins and nucleic acids), cells, and cell aggregates. Michael-type addition of unsaturated groups can take place in good quantitative yields at room or body temperature and under mild conditions with a wide variety of Michael-donors (see, for example, U.S. Ser. No. 09/496,231, U.S. Ser. No. 09/586,937, and U.S. Ser. No. 10/047,404). Furthermore, this reaction can be easily performed in an aqueous environment, e.g., in vivo. Michael-acceptors, such as vinyl sulfones or acrylamides, can be used to link PEG or polysaccharides to proteins through Michael-type reactions with amino- or mercapto-groups; acrylates and many other unsaturated groups can be reacted with thiols to produce cross-linked materials for a variety of biological applications. The reaction of thiols at physiological pH with Michael-acceptor groups shows negligible interference by nucleophiles (mainly amines) present in biological samples. One of the important characteristics of the Michael-type addition reaction as employed in the present methods is its selectivity, i.e. it lacks substantial side reactivity with chemical groups found extracellularly on proteins, cells, and other biological components.

Free Radical Photopolymerization

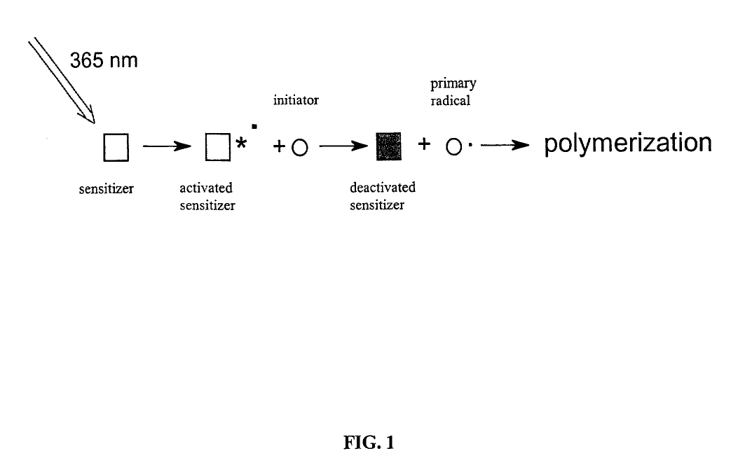

Photopolymerization is another type of reaction that can be used for the curing phase. As is shown in FIG. 1, this reaction involves the free radical polymerization of unsaturated monomers in the presence of a sensitizer and an initiator, or a single molecule acting as both a sensitizer and initiator, under the action of UV or visible light. The free radical photopolymerization of monomers containing more than one reacting group, such as acrylates or acrylamides, yields cross-linked materials that have a negligible content of leachable substances. Because of its high speed (completion in 2-3 minutes), this reaction can be successfully employed in the synthesis of biomaterials (see, for example, Pathak et al., Journal of the American Chemical Society 114:8311-8312 (1992); Mathur et al., Journal of Macromolecular Science-Reviews in Macromolecular Chemistry and Physics, C36:405-430 (1996); Moghaddam et al., Journal of Polymer Science: Part A: Polymer Chemistry 31:1589-1597 (1993); and Zhoa et al., Polymer Preprints 38:526-527 (1997)). The selectivity of reactions that may be achieved with the free-radical photopolymerization reactions may be less than that obtained with the Michael-type addition reactions, described above.

The sensitizer can be any dye which absorbs light having a frequency between 320 nm and 900 nm, is able to form free radicals, is at least partially water soluble, and is non-toxic to the biological material at the concentration used for polymerization. There are a large number of sensitizers suitable for applications involving contact with biological material. Examples of sensitizers include dyes such as ethyl eosin, eosin Y, fluorescein, 2,2-dimethoxy-2-phenyl acetophenone, 2-methoxy, 2-phenylacetophenone, camphorquinone, rose bengal, methylene blue, erythrosin, phloxime, thionine, riboflavin, methylene green, acridine orange, xanthine dye, and thioxanthine dyes. The dyes bleach after illumination and reaction with amines into a colorless product, allowing further beam penetration into the reaction system. Suitable initiators include, but are not limited to, nitrogen based compounds capable of stimulating the free radical reaction, such as triethanolamine, triethylamine, ethanolamine, N-methyl diethanolamine, N,N-dimethyl benzylamine, dibenzyl amine, N-benzyl ethanolamine, N-isopropyl benzylamine, tetramethyl ethylenediamine, potassium persulfate, tetramethyl ethylenediamine, lysine, ornithine, histidine, and arginine.

Examples of the dye/photoinitiator system include, but are not limited to, ethyl eosin with an amine, eosin Y with an amine, 2,2-dimethoxy-2-phenoxyacetophenone, 2-methoxy-2-phenoxyacetophenone, camphorquinone with an amine, and rose bengal with an amine.

In some cases, the dye, such as 2,2-dimethoxy-2-phenylacetophenone, may absorb light and initiate polymerization, without any additional initiator such as the amine. In these cases, only the dye and the precursor components need be present to initiate polymerization upon exposure to the appropriate wavelength of light. The generation of free radicals is terminated when the light source is removed.

The light for photopolymerization can be provided by any appropriate source able to generate the desired radiation, such as a mercury lamp, longwave UV lamp, He-Ne laser, or an argon ion laser. Fiber optics may be used to deliver light to the precursor. Appropriate wavelengths are, for example, within the range of 320-800 nm, such as about 365 nm or 514 nm.

Suitable systems for free radical photopolymerization are well-known in the art and are described in, for example, U.S. Pat. No. 5,858,746 and U.S. Pat. No. 5,801,033.

Structure of the Reactive Groups

Reactive electrophilic groups for Michael-type addition are typically double bonds conjugated with electron withdrawing groups, such as carbonyl, carboxyl and sulfone functionalities:

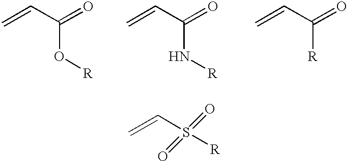

In the above structures, R represents a polymer precursor and the double bonds may optionally be substituted and/or have a ring structure. The substituents on the double bonds can vary the reaction rate by more than one order of magnitude, e.g. poly(ethylene glycol) acrylate reacts roughly ten times faster than the analogous methacrylate and a hundred times faster than the analogous 2,2-dimethylacrylate. Examples of suitable Michael-acceptor groups include, but are not limited to, acrylates, acrylamides, quinones, maleimides, vinyl sulfones, and vinyl pyridiniums (e.g., 2- or 4- vinyl pyridinium).

Thiols or groups containing thiols are exemplary nucleophiles for Michael-type addition reactions. Their reactivity during the Michael-type reaction depends on the thiol pKa. At physiological pH, there is a difference of up to one order of magnitude in the reaction rate of a thiol-containing peptide with acrylic groups if it surrounded by two positive charges or by two negative charges. The incorporation of peptides or proteinaceous material is envisaged mainly in order to obtain a proteolytically degradable material or for specific recognition processes within it (see, e.g., U.S. Ser. No. 10/047,404). Reactions involving thiols containing multiple ester groups are envisaged mainly in order to obtain a hydrolytically degradable material.

Reactive groups for free radical photopolymerization can be, for example, acrylic and methacrylic esters and amides, or styrenic derivatives. Other suitable reactive groups, e.g., ethylenically unsaturated groups, can be employed for photopolymerization.

Preparation of the Polymeric Precursors

The polymeric precursors utilized in this invention can be prepared by direct reaction of functional polymers. Pluronic polymers terminated with OH groups can be converted to acrylates by reaction with acryloyl chloride and provide a polymeric precursor having Michael-acceptor and thermosensitive properties (see Example 2(a) and Scheme 3). These polymers can be further functionalized by Michael-type reaction with an excess of a multifunctional thiol, providing polymeric precursors with Michael-donor and thermosensitive properties (see Example 2(b) and Scheme 3). The acrylated Pluronics can be also used in free radical photopolymerization.

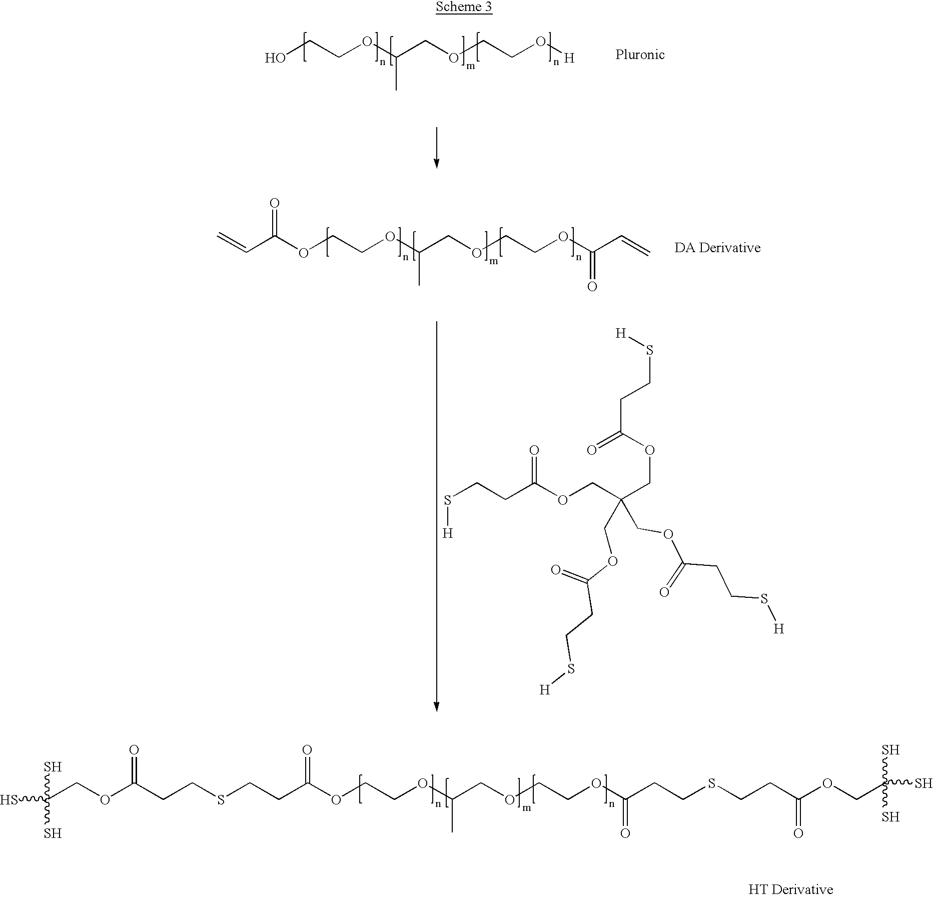

Other polymeric precursors can be prepared following the same scheme from thermosensitive polymers characterized by the presence of functional groups as end groups or in the side chains, such as random or block copolymers of N-isopropylacrylamide and N-hydroxypropylacrylamide obtained by conventional or controlled radical polymerization. A multifunctional Michael-acceptor polymeric precursor can be obtained by reaction of this polymer with acryloyl chloride (Scheme 4). A multifunctional Michael-donor polymeric precursor can be obtained by reaction of the acrylated polymer with an excess of a di- or multithiol, e.g. analogous to the second reaction of Scheme 3.

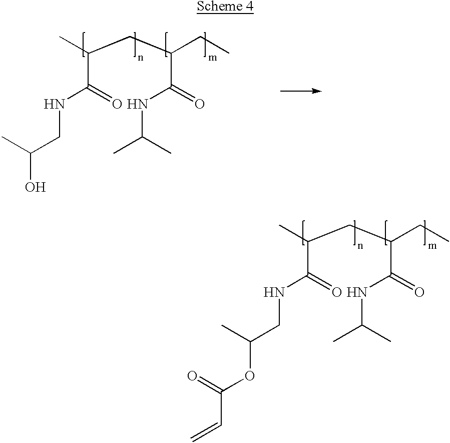

Therapeutic Uses

Since the biomaterials of the present invention can be formed in relatively mild conditions with regard to solvent system, temperature, exothermicity, and pH, and the precursors and products are substantially non-toxic, these materials are suitable for contact with sensitive biological materials, including cells or tissues, and can be used for implantation or other contact with the body. The cross-linking via the Michael-type addition reaction has the potential to be highly self-selective, giving insignificant side reactions with biological molecules, including most macromolecular and small molecule drugs, as well as the molecules on the surfaces of cells to be encapsulated. The gels produced according to the method of the invention have myriad biomedical applications. These applications include but are not limited to drug delivery devices, materials for cell encapsulation and transplantation, barrier applications (adhesion preventatives, sealants), tissue engineering and wound healing scaffolds, materials for surgical augmentation of tissues, and materials for sealants and adhesives.

In one embodiment, the gels are used in biological or drug delivery systems, e.g. for delivery of a bioactive molecule. A bioactive molecule may be any biologically active molecule, for example, a natural product, synthetic drug, protein (such as growth factors or enzymes), or genetic material. The carrier must preserve the functional properties of such a bioactive molecule. The bioactive molecule may be released by diffusive mechanisms or by degradation of the gel carrier through a variety of mechanisms (such as hydrolysis or enzymatic degradation) or by other sensing mechanisms (for example, pH induced swelling). Given that many bioactive molecules contain reactive groups, it is important that the material that serves as the carrier not react with the bioactive molecules in an undesirable manner; as such, the high self-selectivity of reactions between conjugated unsaturations and thiols is very useful in drug encapsulation. In regard to the encapsulation of hydrophobic molecules, e.g. hydrophobic drugs, the hydrophobic domains created in the gel material as a result of the presence of the hydrophobic parts of the copolymers that lead to the thermal gelation may be useful as hydrophobic nano- and microdomains to serve as sites for physicochemical partitioning of the drug to lead to more sustained release.

The biomaterials of the invention also have biomedical applications as encapsulation and transplantation devices. Such devices serve to isolate cells (e.g., allograft or xenograft) from a host's defense system (immunoprotect) while allowing selective transport of molecules such as oxygen, carbon dioxide, glucose, hormones, and insulin and other growth factors, thus enabling encapsulated cells to retain their normal functions and to provide desired benefits, such as the release of a therapeutic protein that can diffuse through the immunoprotection hydrogel membrane to the recipient.

Because of the biocompatibility of the biomaterials and techniques involved, in part due to the self-selectivity of the cross-linking chemistries, a wide variety of biologically active substances and other materials can be encapsulated or incorporated, including, but not limited to, proteins, peptides, polysaccharides, organic or inorganic drugs, nucleic acids, sugars, cells, and tissues.

Examples of cells, which can be encapsulated, are primary cultures as well as established cell lines, including transformed cells. These include, but are not limited to, pancreatic islet cells, human foreskin fibroblasts, Chinese hamster ovary cells, beta cell insulomas, lymphoblastic leukemia cells, mouse 3T3 fibroblasts, dopamine secreting ventral mesencephalon cells, neuroblastoid cells, adrenal medulla cells, and T-cells. As can be seen from this partial list, cells of all types, including dermal, neural, blood, organ, muscle, glandular, reproductive, and immune system cells can be encapsulated successfully by this method. Additionally, proteins (such as hemoglobin), polysaccharides, oligonucleotides, enzymes (such as adenosine deaminase), enzyme systems, bacteria, microbes, vitamins, cofactors, blood clotting factors, drugs (such as TPA, streptokinase or heparin), antigens for immunization, hormones, and retroviruses for gene therapy can be encapsulated by these techniques.

Biomaterials for use as scaffolds are desirable for tissue engineering and wound healing applications: nerve regeneration, angiogenesis, and skin, bone, and cartilage repair and regeneration. Such scaffolds may be introduced to the body pre-seeded with cells or may depend upon cell infiltration from outside the material in the tissues near the implanted biomaterial. Such scaffolds may contain (through covalent or non-covalent bonds) cell interactive molecules like adhesion peptides and growth factors.

The biomaterials of the invention can also be used as materials for coating cells, tissues, microcapsules, devices, and other implants. The shape of such an implant can match the tissue topography, and a relatively large implant can be delivered through minimally invasive methods.

The present invention is illustrated by the following examples that describe the methods and compositions of the invention. The examples are provided for the purpose of illustrating the invention, and are in no way intended to be limiting of the invention.

### EXAMPLE 1

### Thermal Gelation of Pluronic Block Copolymers

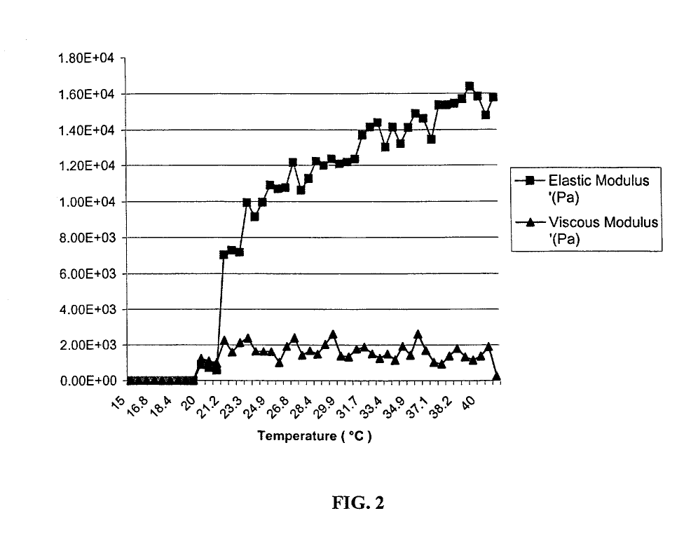

0.5 g of solid pluronic F127 were dispersed in 2 g of distilled water and the mixture was left in an ice bath (0° C.) for 2 hours until complete dissolution. 50 μl of cold polymer solution (20% wt/wt) were transferred to a parallel plate rheometer and carefully overlaid with a low viscosity silicon oil to minimize water evaporation. The rheometer was used in oscillatory mode, where the outer plate performs sinusoidal oscillation at given frequency (0.5 Hz) and given stress (20 Pa), according to the linear viscoelastic region of the material. The temperature was varied from 10° C. to 40° C. in increments of 1° C. with 4 min equilibration time at each step. Elastic and viscous modulus increased with temperature at different rates; the gelation point (recorded as the crossing of the elastic and viscous modulus lines) was recorded at 19° C. (FIG. 2)

### EXAMPLE 2

### Preparation of Reactive Pluronic Derivatives

(a) Preparation of Pluronic F-127 Diacrylate (F127DA).

25 g Pluronic F127 were dissolved in 250 ml of toluene and dried with molecular sieves under reflux in a Soxhlet apparatus for 3 hours. After cooling to 0° C., 50 ml of dichloromethane and 1.66 ml of triethylamine (12 mmol) were added under argon. 0.64 ml of acryloyl chloride (7.9 mmol) were dropped into the reaction mixture, and the solution was left for 6 hours under stirring. The mixture was then filtrated, concentrated at the rotatory evaporator, diluted with dichloromethane and extracted with distilled water two times. The dichloromethane solution was dried with sodium sulphate and then precipitated in n-hexane.

(b) Preparation of Pluronic F-127 Hexathiol (F127HT).

4 g of F127DA (pluronic F127 diacrylate) and 1.55 g (molar ratio thiol/acrylate ˜10:1) of pentaerythritol tetrakis (3-mercaptopropionate) (QT) were dissolved in 50 ml of 1-methyl-2-pyrrolidone (NMP). Drops of NaOH 0.1 M were added until the pH of the solution increased to 9. The reaction mixture, previously degassed by argon bubbling, was left under argon atmosphere and stirring overnight at room temperature. The solution was then concentrated at the rotatory evaporator using a high vacuum pump (p=0.3 mbar), diluted in dichloromethane, and extracted with distilled water two times. The dichloromethane solution was dried with sodium sulphate and then precipitated in cold diethyl ether. The dry polymer was redissolved in 25 ml of NMP adding 40 mg of 1,4-Dithio-DL-threitol (DTT). The solution was stirred under argon for 15min and then precipitated in cold diethyl ether. 3.8 g of colorless material were recovered.

### EXAMPLE 3

### Curing Without Thermal Gelation of Reactive Pluronic Derivatives

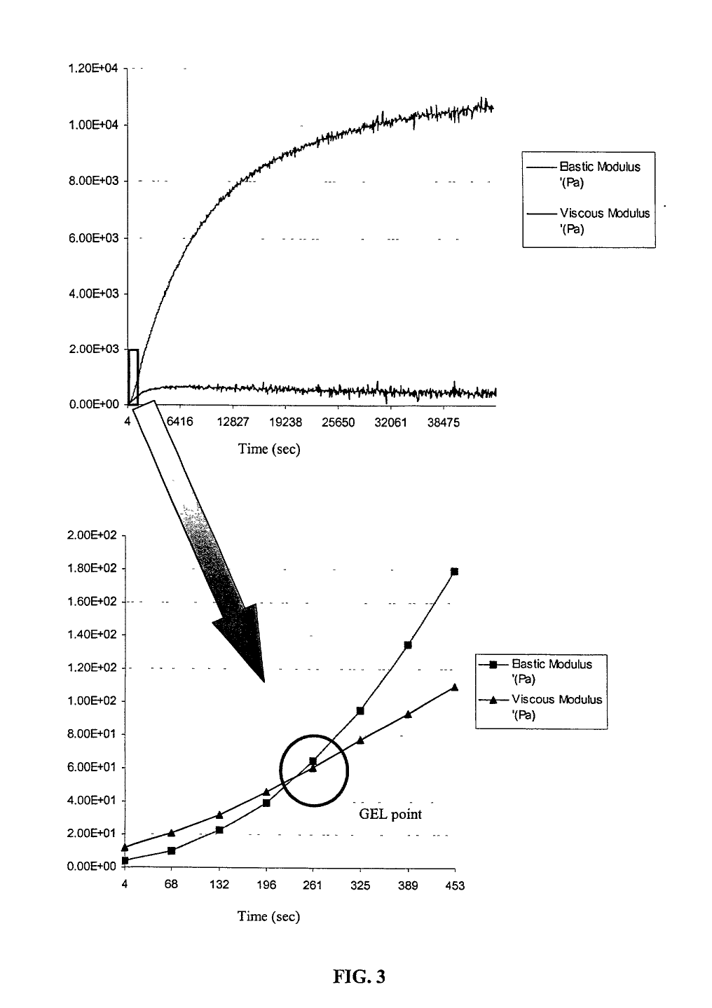

0.185 g of solid F127DA and 0.065 g of solid F127HT were dispersed in 2 g of PBS pH=7.4, and the mixture was left in an ice bath (0° C.) for 2 hours until complete dissolution. The cold polymer solution (11% wt/wt) was transferred to the rheometer, previously cooled at 5° C. The temperature was then quickly increased until 37° C., and the oscillation test was started (frequency 0.5 Hz, stress 20 Pa) keeping the temperature at 37° C. The gelation point (recorded as the crossing of the elastic and viscous modulus lines) was recorded after 260 sec, while the elastic modulus reached a plateau (corresponding to a value of 10-12 kPa) after a few hours (FIG. 3).

### EXAMPLE 4

### Curing With Thermal Gelation of Reactive Pluronic Derivatives

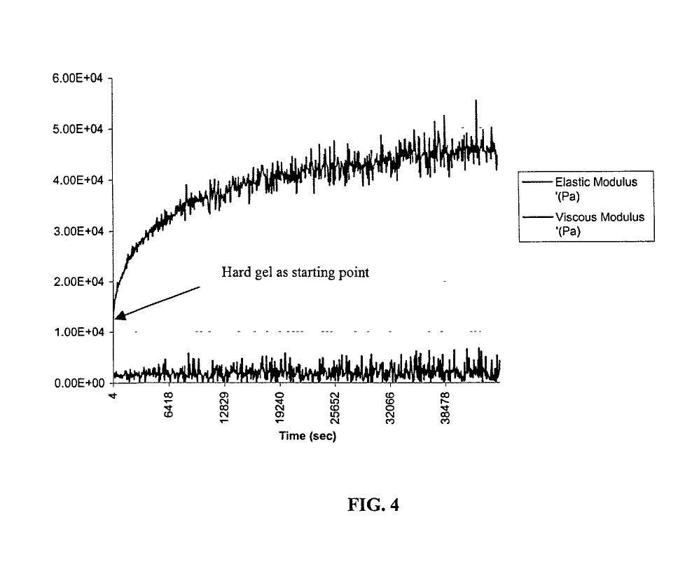

0.37 g of solid F127DA and 0.13 g of solid F127HT dispersed in 2 g of PBS pH=7.4, and the mixture was left in an ice bath (0° C.) for 2 hours until complete dissolution. The cold polymer solution (20% wt/wt) was transferred to the rheometer, previously cooled at 5° C. The temperature was then quickly increased until 37° C., and the oscillation test was started (frequency 0.5 Hz, stress 20 Pa) keeping the temperature at 37° C. At the beginning of the measurement, the elastic modulus was higher than the viscous modulus, indicating that thermal gelation had already occurred; the curing reaction caused an increase of the elastic modulus, reaching a plateau of 40-50 kPa after 10 hours (FIG. 4).

### EXAMPLE 5

### Bead Formation

0.37 g of solid F127DA and 0.13 g of solid F127HT were dispersed in 2 g of PBS 10 mM pH=7.4, and the mixture was left in an ice bath (0° C.) for 2 hours under stirring. The cold polymer solution (20% wt/wt, pH˜7) was transferred into a syringe (25G1 needle) and was dropped in a bath solution (Dulbecco's MEM+Fetal Bovinum Serum 10%) at 37° C. The droplets were instantly solidified in the bath (thermal gelation) and the curing phase was completed after 12 hours standing in the incubator at 37° C. The beads had an average diameter of 3 mm.

This procedure can be accomplished in commercial encapsulators to give sub-mm beads, whose diameter can be regulated with the help of a vibrating nozzle.

Gelation can be performed in presence of biological materials, such as cells, enzymes, and drugs. The biological material may be dispersed in the polymeric precursor solution. Alternatively, the gelling solution can also be extruded through the outer space of a double nozzle construct, where a biological material is extruded in a non-gelling solution through the internal one; in this way, capsules are generated where the biological material is contained in a water non-gelled internal cavity and are surrounded by a spherical membrane.

### EXAMPLE 6

### Tubing Formation

0.37 g of solid F127DA and 0.13 g of solid F127HT were dispersed in 2 g of PBS 10 mM pH=7.4, and the mixture was left in an ice bath (0° C.) for 2 hours under stirring. The cold polymer solution (20% wt/wt, pH˜7) was transferred into a mold made of a cylinder equipped with an internal pistol (e.g. a stopped syringe), kept at 37° C. The gel formed instantaneously and could be immediately recovered; the curing phase was completed after incubation at 37° C. for 12 hours.

Tubes can be produced also through a double nozzle extruder, where a warmer fluid (water, air) flows through the internal space; the solution thermally gels when comes in direct contact with the warmer fluid and produces a hollow cylindrical construct. The warmer fluid can contain biologically active materials and thus allow the encapsulation of cells, enzymes or drugs in a non-spherical construct.

### Other Embodiments

Although the present invention has been described with reference to preferred embodiments, one skilled in the art can easily ascertain its essential characteristics and, without departing from the spirit and scope thereof, can make various changes and modifications of the invention to adapt it to various usages and conditions. Those skilled in the art will recognize or be able to ascertain using no more than routine experimentation, many equivalents to the specific embodiments of the invention described herein. Such equivalents are intended to be encompassed in the scope of the present invention.

All publications, patents, and patent applications, mentioned in this specification are hereby incorporated by reference.

We claim:

1. A method for preparing a biomaterial, said method comprising the steps of:
(a) providing a polymeric precursor comprising reactive groups, wherein said polymeric precursor undergoes reverse thermal gelation in aqueous solution;
(b) shaping said polymeric precursor by thermally inducing gelation of an aqueous solution of said polymeric precursor; and
(c) curing said polymeric precursor by cross-linking said reactive groups to produce said biomaterial.

2. The method of claim 1 where the polymeric precursor is a polyether or a block copolymer, wherein in at least one of the blocks is a polyether, poly(N-alkyl acrylamide), hydroxypropylcellulose, poly(vinylalcohol), poly(ethyl(hydroxyethyl)cellulose), polyoxazoline, or a derivative thereof containing reactive groups in side chains or as terminal groups.

3. The method of claim 1, wherein said curing step (b) comprises cross-linking said polymeric precursor using a Michael-type addition reaction.

4. The method of claim 3, wherein said Michael-type reaction is characterized by the nucleophilic addition of a thiol and a Michael-acceptor selected from the group consisting of acrylates, acrylamides, quinones, maleimides, vinyl sulfones, or vinyl pyridiniums.

5. The method of claim 1, wherein said curing step (b) comprises cross-linking said polymeric precursor using a radical photopolymerization reaction.

6. The method of claim 5, wherein said photopolymerization reaction occurs in the presence of a sensitizer and an initiator.

7. The method of claim 6, wherein said sensitizer is selected from the group consisting of ethyl eosin, eosin Y, fluorescein, 2,2-dimethoxy-2-phenyl acetophenone, 2-methoxy, 2-phenylacetophenone, camphorquinone, rose bengal, methylene blue, erythrosin, phloxime, thionine, riboflavin, methylene green, acridine orange, xanthine dye, and thioxanthine dyes

8. The method of claim 6, wherein said initiator is selected from the group consisting of triethanolamine, triethylamine, ethanolamine, N-methyl diethanolamine, N,N-dimethyl benzylamine, dibenzyl amine, N-benzyl ethanolamine, N-isopropyl benzylamine, tetramethyl ethylenediamine, potassium persulfate, tetramethyl ethylenediamine, lysine, ornithine, histidine, and arginine.

9. A biocompatible gel prepared by the method of:
(a) providing a polymeric precursor comprising reactive groups, wherein said polymeric precursor undergoes reverse thermal gelation in aqueous solution;
(b) shaping said polymeric precursor by thermally inducing gelation of an aqueous solution of said polymeric precursor; and
(c) curing said polymeric precursor by cross-linking said reactive groups using a Michael-type addition reaction to produce said biomaterial.

10. The gel of claim 9, wherein said shaping in step (b) produces capsules or beads.

11. The gel of claim 9, wherein said shaping in step (b) produces tubes, hollow fibers, or solid fibers.

12. The gel of claim 9, further comprising a bioactive molecule or a cell.

13. The gel of claim 12, wherein said bioactive molecule is selected from the group consisting of protein, naturally occurring or synthetic molecules, viral particles, sugars, polysaccharides, organic or inorganic drugs, and nucleic acid molecules.

14. The gel of claim 12, wherein said cell is selected from the group consisting of pancreatic islet cells, human foreskin fibroblasts, Chinese hamster ovary cells, beta cell insulomas, lymphoblastic leukemia cells, mouse 3T3 fibroblasts, dopamine secreting ventral mesencephalon cells, neuroblastoid cells, adrenal medulla cells, and T-cells.

15. A drug delivery vehicle comprising:
(a) a gel produced by the method of:
(i) providing a polymeric precursor comprising reactive groups, wherein said polymeric precursor undergoes reverse thermal gelation in aqueous solution;
(ii) shaping said polymeric precursor by thermally inducing gelation of an aqueous solution of said polymeric precursor; and
(iii) curing said polymeric precursor by cross-linking said reactive groups using a Michael-type addition reaction to produce said biomaterial; and
(b) a therapeutic substance.

16. The delivery vehicle of claim 15, wherein said therapeutic substance is selected from the group consisting of synthesized organic molecules, naturally occurring organic molecules, nucleic acids, biosynthetic peptides, naturally occurring peptides, and modified peptides.

17. A method for delivering a therapeutic substance to a cell, tissue, organ, organ system, or body of an animal said method comprising the steps of:
(a) providing a drug delivery vehicle comprising a therapeutic substance and a gel produced by the method of:
(i) providing a polymeric precursor comprising reactive groups, wherein said polymeric precursor undergoes reverse thermal gelation in aqueous solution;
(ii) shaping said polymeric precursor by thermally inducing gelation of an aqueous solution of said polymeric precursor; and
(iii) curing said polymeric precursor by cross-linking said reactive groups using a Michael-type addition reaction to produce said biomaterial; and
(b) contacting said cell, tissue, organ, organ system or body with said drug delivery system.

18. The method of claim 17, wherein said therapeutic substance is selected from the group consisting of proteins, naturally occurring or synthetic organic molecules, viral particles, and nucleic acid molecules.

19. The method of claim 17, wherein said therapeutic substance is a prodrug.

20. The method of claim 17, wherein said nucleic acid molecule is an antisense nucleic acid molecule.

21. A biocompatible gel prepared by the method of:
(a) providing a polymeric precursor comprising reactive groups, wherein said polymeric precursor undergoes reverse thermal gelation in aqueous solution;
(b) shaping said polymeric precursor by thermally inducing gelation of an aqueous solution of said polymeric precursor; and
(c) curing said polymeric precursor by cross-linking said reactive groups using a radical photopolymerization reaction to produce said biomaterial.

22. The gel of claim 21, wherein said shaping in step (b) produces capsules or beads.

23. The gel of claim 21, wherein said shaping in step (b) produces tubes, hollow fibers, or solid fibers.

24. The gel of claim 21, further comprising a bioactive molecule or a cell.

25. The gel of claim 24, wherein said bioactive molecule is selected from the group consisting of proteins, naturally occurring or synthetic organic molecules, viral particles, sugars, polysaccharides, organic or inorganic drugs, and nucleic acid molecules.

26. The gel of claim 24, wherein said cell is selected from the group consisting of pancreatic islet cells, human foreskin fibroblasts, Chinese hamster ovary cells, beta cell insulomas, lymphoblastic leukemia cells, mouse 3T3 fibroblasts, dopamine secreting ventral mesencephalon cells, neuroblastoid cells, adrenal medulla cells, and T-cells.

27. A drug delivery vehicle comprising:
(a) a gel produced by the method of:
(i) providing a polymeric precursor comprising reactive groups, wherein said polymeric precursor undergoes reverse thermal gelation in aqueous solution;
(ii) shaping said polymeric precursor by thermally inducing gelation of an aqueous solution of said polymeric precursor; and
(iii) curing said polymeric precursor by cross-linking said reactive groups using a radical photopolymerization reaction to produce said biomaterial; and
(b) a therapeutic substance.

28. The delivery vehicle of claim 27, wherein said therapeutic substance is selected from the group consisting of synthesized organic molecules, naturally occurring organic molecules, nucleic acids, biosynthetic peptides, naturally occurring peptides, and modified peptides.

29. A method for delivering a therapeutic substance to a cell, tissue, organ, organ system, or body of an animal said method comprising the steps of:
(a) providing a drug delivery vehicle comprising a therapeutic substance and a gel produced by the method of:
(i) providing a polymeric precursor comprising reactive groups, wherein said polymeric precursor undergoes reverse thermal gelation in aqueous solution;
(ii) shaping said polymeric precursor by thermally inducing gelation of an aqueous solution of said polymeric precursor; and
(iii) curing said polymeric precursor by cross-linking said reactive groups using a radical photopolymerization reaction to produce said biomaterial; and
(b) contacting said cell, tissue, organ, organ system or body with said drug delivery system.

30. The method of claim 29, wherein said therapeutic substance is selected from the group consisting of proteins, naturally occurring or synthetic organic molecules, viral particles, and nucleic acid molecules.

31. The method of claim 29, wherein said therapeutic substance is a prodrug.

32. The method of claim 30, wherein said nucleic acid molecule is an antisense nucleic acid molecule.
